(19) United States
(12) Patent Application Publication
(10) Pub. No.: US 2025/0242143 A1
(43) Pub. Date: Jul. 31, 2025

(54) VASCULAR ACCESS SYSTEM

(71) Applicant: VERIVAS SOLUTIONS INC., Stamford, CT (US)

(72) Inventor: Ross Traut Lyon, Fontana, WI (US)

(73) Assignee: VERIVAS SOLUTIONS INC., Stamford, CT (US)

(21) Appl. No.: 18/844,425

(22) PCT Filed: Mar. 7, 2022
(86) PCT No.: PCT/US2022/019204

§ 371 Date: Sep. 6, 2024

(51) Int. Cl. A61M 39/02; A61M 39/06

(52) U.S. Cl. CPC A61M 39/0247; A61M 39/06; A61M 2039/0258; A61M 2039/027; A61M 2039/0279; A61M 2039/0288; A61M 2039/062; A61M 2039/0626

(57) ABSTRACT

A vascular access system for repeatedly accessing a blood vessel includes a sheathed access conduit that remains outside of the confines of the vascular system. The tubular access conduit includes a first end adapted to be arranged outside the body for easy access and a second end adapted to be connected to an outer surface of the blood vessel. A stent is encapsulated inside the subcutaneously portion of the access conduit to avoid contracture. A vessel entry device is adapted to extend inside the access conduit to puncture and to gain access to the blood vessel lumen when needed, otherwise the conduit remains excluded from the vascular system. Guidewires, catheter, monitors or treatment devices can be inserted, advanced, positioned, used and removed as desired via the access conduit. Because the conduit remains external to the blood vessel the risks of septicemia and vessel thrombosis and occlusion are reduced.

### CROSS-REFERENCE TO RELATED APPLICATIONS

This application claims priority from U.S. Provisional Application No. 63/157,852 filed Mar. 8, 2021, titled “Verivas Dialysis Access Conduit”, the content of which is incorporated herein.

### FIELD OF THE PRESENT DISCLOSURE

The present disclosure relates generally to a medical apparatus, and more specifically, to a vascular access system that enables repeated access of a blood vessel lumen for performing medical procedures, for example, treatments or sampling or monitoring or any other medical procedures.

### BACKGROUND

Hemodialysis and other percutaneous vascular procedures are some of the most commonly performed invasive medical procedures. Vascular access for these procedures currently utilizes vascular conduits that are in direct continuity with a blood vessel lumen or reside, at least partially, within the blood vessel lumen. As a result, these devices have significant risks of bleeding, clotting, infection and other serious problems requiring frequent hospitalization, invasive treatments and reoperation.

Vascular access systems having a vascular access device that resides outside the blood vessel are known in the art. Such vascular access devices generally include a tubular structure having one or more ends attached to the surface of the blood vessel and other end arranged distally from the blood vessel and with the internal lumen of the device in direct continuity with the internal lumen of a blood vessel at both ends of the device, thereby allowing continuous blood flow through the device. Such devices normally reside inside the subcutaneous tissue, require high internal blood flow rates and are often difficult to access from outside the body, requiring percutaneous punctures to do so. Accordingly, such devices commonly fail as a result of poor flow or repeated punctures required for access.

### SUMMARY

According to some aspects of the disclosure, a vascular access system for accessing a blood vessels of a body is disclosed. The vascular access system includes an access device having an access conduit and a stent. The access conduit has a first end adapted to be arranged outside the body and the other opposite second end, with an embedded stent, resides inside the body and is attached to the external surface of a blood vessel but is not in direct continuity with the blood vessel lumen. The conduit thereby conveys direct access to the surface of a blood vessel from outside the body. The first end is adapted to allow easy external access whereas the second end is adapted to be arranged inside the body to facilitate directed access, into the lumen of a blood vessel. The stent prevents collapsing of the indwelling portion of the access conduit. Because the conduit device resides entirely outside of and is not in continuity with the lumen of a blood vessel, it is not as prone to bleeding, clotting or infection entering the blood stream. The vascular access system also includes a vessel entry device adapted to extend, at least partially, from the outside end to inside end of the access conduit in order to puncture and thereby gain access to the blood vessel lumen when needed. Once blood vessel entry via the access conduit and the puncture device has been achieved, a directional guidewire can be partially introduced and advanced in the blood vessel lumen to the appropriate vascular location to enable placement and or delivery of a variety of medical devices, medications or performance of medical diagnostic or therapeutic procedures or treatments. When the procedures or necessary vascular treatments are completed, the devices inserted via the conduit can be removed thereby allowing the opening from the conduit into the blood vessel to close and heal over, thereby re-establishing a tissue barrier between the conduit and the lumen of the blood vessel.

In one aspect, a vascular access system for accessing a blood vessel of a body is disclosed. The vascular access system includes an access device having an access conduit and a stent. The access conduit has a first end and a second end adapted to be arranged outside the blood vessel and connected to an outer surface of the blood vessel. The access conduit also includes a first longitudinal portion extending from the first end towards the second end and adapted to be arranged outside the body to facilitate an access of the access conduit, and a second longitudinal portion extending from the first longitudinal portion to the second end and adapted to be arranged inside the body. The stent is encapsulated inside the second longitudinal portion of the access conduit to prevent collapsing of the access conduit. The vascular access system also includes a vessel entry device adapted to extend, at least partially, inside the access conduit, allowing only the specialized puncture tip portion of the vessel puncture device to puncture and gain access to the lumen of the blood vessel. The vessel entry device has a cylindrical body defining a central lumen extending from a first end to a second end of the cylindrical body, and a needle coaxially arranged relative to the cylindrical body and extending outwardly from the second end of the cylindrical body. The needle includes a puncture tip to puncture and thereby gain access to the lumen of the blood vessel. The needle and puncture tip maybe straight or curved. Specifically designed right angle shoulders are constructed at the junction of the body of the vessel entry device and the protruding vessel puncture tip, to prevent all but the puncture tip from entering the blood vessel lumen.

In some embodiments, the access conduit includes a sheath wrapped around an outer surface of the access conduit and extending at least along a portion of a length of the access conduit.

In some embodiments, the entry device includes a pair of grab arms arranged at the first end of the cylindrical body and extending radially outwardly from the cylindrical body.

In some embodiments, the vascular access system further includes a valve adapted to be attached to the access conduit and cover a first of the access conduit. The valve includes a flexible membrane biased in a closed position to close the first port of the access conduit.

In some embodiments the vessel entry device is inserted inside the access conduit through the flexible membrane of the valve to puncture the blood vessel.

In some embodiments, the valve includes a cylindrical portion defining a central passageway, wherein an outer surface of the cylindrical portion defines external threads adapted to facilitate the attachment of the valve with the access conduit.

In some embodiments, the flexible membrane is arranged at one end of the cylindrical portion to close the central passageway.

In some embodiments, the vascular access system includes a cap adapted to extend, at least partially, inside the access conduit to close the access conduit. The cap is attached to the access conduit upon completion of the medical procedure.

In another aspect, a vascular access system for accessing a blood vessel of a body is disclosed. The vascular access system includes an access device having an access conduit and a stent. The access conduit has a first end and a second end adapted to be arranged outside the blood vessel and connected to an outer surface of the blood vessel. The access conduit also includes a first longitudinal portion extending from the first end towards the second end and adapted to be arranged outside the body to facilitate an access of the access conduit, and a second longitudinal portion extending from the first longitudinal portion to the second end and adapted to be arranged inside the body. The stent is encapsulated inside the second longitudinal portion to prevent a collapsing of the conduit.

In some embodiments, the vascular access system further includes a vessel entry device adapted to extend, only partially, inside the access conduit to selectively puncture only the adjacent wall of the blood vessel so as to gain access the vessel lumen. The vessel entry device includes a cylindrical body defining a central lumen extending from a first end to a second end of the cylindrical body, and a vessel lumen entry needle coaxially arranged relative to the cylindrical body and extending outwardly from the second end of the cylindrical body with right angles shoulders at the junction of the body (of the entry device) with the entry needle to keep the body of the entry device from entering the lumen of the blood vessel. The needle includes a puncture tip to puncture and enter the lumen of the blood vessel. The needle and puncture tip may be either straight or curved.

In some embodiments, the access conduit includes a sheath wrapped around an outer surface of the access conduit and extending at least along a portion of a length of the access conduit.

In some embodiments the access conduit is coated along its surfaces(s) with various chemicals, substances or components to reduce the risk of infection, clotting or obstruction.

In some embodiments, the vascular access system further includes a valve adapted to be attached to the access conduit to selectively provide an access of the access conduit. The valve includes a flexible membrane biased in a closed position to close a first port of the access conduit.

In some embodiments, the vessel entry device is inserted inside the access conduit through the flexible membrane of the valve and advanced only enough for the tip of the entry needle to puncture the adjacent single blood vessel wall and enter the blood vessel lumen without the body of the vessel entry device entering the lumen of the blood vessel.

In some embodiments, the valve includes a cylindrical portion defining a central passageway, wherein an outer surface of the cylindrical portion defines external threads adapted to facilitate the attachment of the valve with the access conduit.

In some embodiments, the flexible membrane(s) is arranged at one end of the cylindrical portion to close the central passageway.

In some embodiments, the vascular access system further includes a cap adapted to extend, at least partially, inside the conduit to close the access conduit. The cap is attached to the access conduit upon completion of the medical procedure.

In yet another aspect, a method for performing a medical procedure is disclosed. The method includes attaching an access device to an outer surface of a blood vessel. The access device includes an access conduit having a first end, a second end arranged opposite to the first end, a first longitudinal portion extending from the first end towards the second end, and a second longitudinal portion extending from the first longitudinal portion to the second end. The access device also includes a stent encapsulated inside the second longitudinal portion of the access conduit to prevent a collapsing of the conduit. The access device is attached to the blood vessel by arranging the first longitudinal portion outside the body and second longitudinal portion inside the body. The second end of the access conduit is attached to the outer surface of the blood vessel. The method also includes engaging a valve at the first end of the access conduit. The valve includes a flexible membrane biased to close a central passage of the access conduit and selectively provides an access of the access conduit. The method also includes inserting a vessel entry device having a needle through the membrane and the central passage of the access conduit to puncture the blood vessel.

In some embodiments, the method further includes extending a guidewire inside the vessel entry device to the blood vessel, wherein guidewire extends from an outside of the valve to the blood vessel through the vessel entry device, and removing the vessel entry device from the access conduit through the valve while retaining the guidewire inside the access conduit. The method also includes inserting a medical device over the guidewire inside the access conduit and extending a portion of the medical device inside the blood vessel through the puncture formed in the blood vessel and advancing the medical device over the guidewire to a desired location of a vascular system of the body in order to properly perform the medical procedure.

In some embodiments, the method further includes removing the medial device and the guidewire upon completion of the medical procedure, disengaging the valve from the access conduit, and engaging a cap to the first end of the access conduit to plug the access conduit.

In some embodiments, the method further includes flushing the access conduit with one or more fluids and/or chemicals before engaging the cap to the access conduit.

The features and advantages described in this disclosure and in the following detailed description are not all-inclusive. Many additional features and advantages will be apparent to one of ordinary skill in the relevant art in view of the drawings, specification, and claims hereof. Moreover, it should be noted that the language used in the specification has been principally selected for readability and instructional purposes and may not have been selected to delineate or circumscribe the inventive subject matter; reference to the claims is necessary to determine such inventive subject matter.

### BRIEF DESCRIPTION OF THE DRAWINGS

For a more complete understanding of example embodiments of the present disclosure, reference is now made to the following descriptions taken in connection with the accompanying drawings in which:

FIG. 1 illustrates a diagrammatic view of an access device of a vascular access system attached to a blood vessel, in accordance with an example embodiment of the present disclosure;

FIG. 2 illustrates a diagrammatic view of an entry device of the vascular access system, in accordance with an example embodiment of the present disclosure;

FIG. 3 illustrates a diagrammatic view of a cap adapted to engage with the access device of FIG. 1, in accordance with an example embodiment of the present disclosure; and

FIG. 4 illustrates a diagrammatic view of a valve of the vascular access system, in accordance with an example embodiment of the present disclosure.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. In other instances, apparatuses and methods are shown in block diagram form only in order to avoid obscuring the present disclosure.

Embodiments of the present invention are hereafter described in detail with reference to the accompanying Figures. Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention.

The following description with reference to the accompanying drawings is provided to assist in a comprehensive understanding of exemplary embodiments of the present invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Like numbers refer to like elements throughout. In the figures, the sizes of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. Thus, for example, a reference to “a component surface” includes reference to one or more of such surfaces.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be also understood that when an element is referred to as being “on,” “attached” to, “connected” to, “coupled” with, “contacting”, “mounted” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of reasonable skill in the relevant art that references to a structure or a feature that is “adjacent” to another feature may have portions that overlap or underlie that feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical, “horizontal” and the like are used herein for the purpose of explanation only unless specifically indicated otherwise.

Included in the description are flowcharts depicting examples of the methodology which may be used to utilize the disclosed apparatus for harvesting of a subcutaneous blood vessel. Accordingly, blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware and/or computer systems that perform the specified functions or steps, or combinations of special purpose hardware and computer instructions.

Upon reading this disclosure, those of skill in the art will appreciate still additional alternative structural and functional designs for the disclosed apparatus for harvesting of a subcutaneous blood vessel through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope of the invention.

The present disclosure provides an apparatus or system for repeatedly access a blood vessel for performing a medical procedure. The present apparatus or system is in the form of a kit including multiple components which works in combination to achieve the given purpose. Hereinafter, the terms “subcutaneous blood vessel,” “blood vessel,” and “vein” have been interchangeably used without any limitations. The vascular access system can be utilize to perform any, but not limited to, of the following: fluid, medication, thermal agents or blood product administration, dialysis, plasmapheresis, venous, arterial or cardiac catheterization procedures, arterial or venous angioplasty, coronary or peripheral vascular stenting or endovascular grafting, cardiac valvular replacement, intra-aortic balloon placement, embolectomy, ablation procedures partial circulatory assist or other vascular, cardiac or cerebral or other procedures or device use facilitated by vascular access.

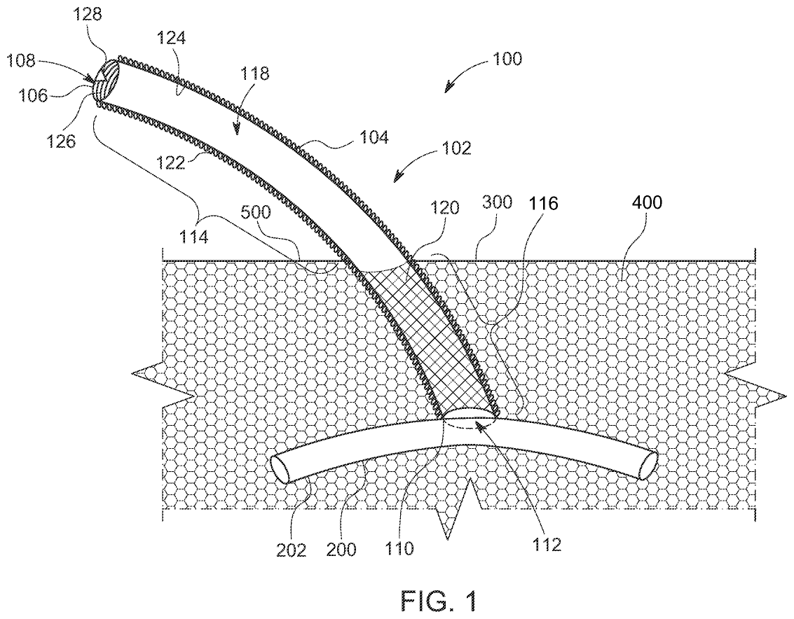

Referring to FIG. 1, a vascular access system 100 for performing a medical procedure, for example, a dialysis, having an access device 102 attached to a blood vessel 200 of a body 300 is shown. The access device 102 includes an access conduit 104, which is an elongated flexible conduit, having a first end 106 defining a first port 108, a second end 110 defining a second port 112, a first longitudinally extending portion 114 extending from the first end 106 towards the second end 110, and a second longitudinally extending portion 116 that extends from the first longitudinal portion 114 to the second end 110. As shown, the conduit 104 defines a central passage 118 extending from the first port 108 to the second port 112 As shown, the second end 110 is arranged outside the blood vessel 200 and is engaged with an outer surface 202 of the blood vessel 200, while the first end 106 is arranged outside the body 300. The second end 110 may be attached to the outer surface 202 of the blood vessel 200 by suturing, and then applying a sealant adhesive to form a leak proof connection/joint between the access conduit 104 and the outer surface 202 of the blood vessel 200 to prevent a leakage of the blood or other fluid, while the medical procedure is being performed.

Further, the first longitudinal portion 114 extends outside the body 300, while the second longitudinal portion 116 is adapted to be arranged inside the body 300, and extends through subcutaneous tissues 400 from the outer surface 202 of the blood vessel 200 to a skin surface 500 of the body 300. To enable the second longitudinal portion 116 of the conduit 104 to reside inside the body 300, the access device 102 includes a stent 120 encapsulated inside a wall of the second longitudinal portion 116 of the conduit 104. The stent 120 facilitates a flexing of the second longitudinal portion 114 to enable the conduit 104 (i.e., the central passage 118) to retain its shape under the influence of compression applied by surrounding subcutaneous tissues 400 on the conduit 104. Further, the access device includes a sheath 122 wrapped around an outer surface of the conduit 104 and extending from the first end 106 up to or alternatively just short of the second end 110. The sheath 122 may be made of a porous fabric and enables tissue growth around the exterior of the conduit 104. Although, the sheath 122 is shown to be wrapped around both the first portion 114 and the second portion 116, it may be appreciated that the sheath 122 may be wrapped/dispose around only the portion of the outer surface of the conduit 104 associated with the second longitudinal portion 116, and the portion of the outer surface associated with the first portion 116 is devoid of the sheath 122. Moreover, the sheath 122 may extend along a part length of the second longitudinal portion 116. Additionally, an inner surface 124 of the conduit 104 defines internal threads 126 arranged proximate to the first end 106 of the access conduit 104 and extending away from the first end 106 towards the second end 110. Moreover, the inner surface 124 of the conduit 104 defines an engagement structure 128, for example, notch or a ridge at the first end 106.

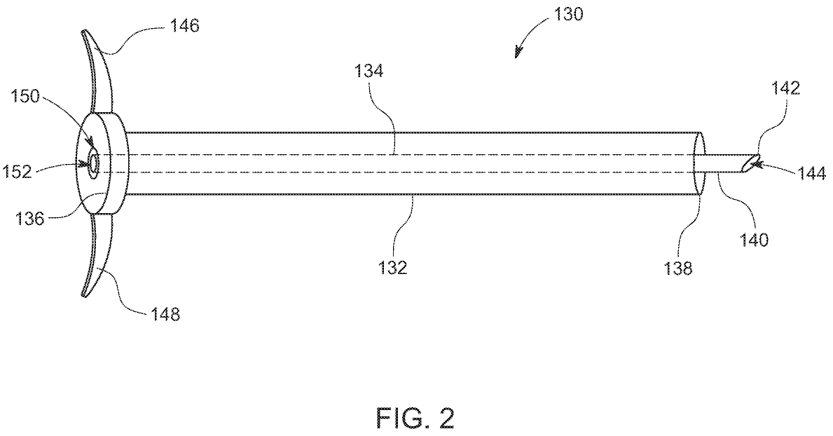

Referring to FIG. 2, the vascular access system 100 further includes a vessel entry device 130 adapted to be inserted inside the conduit 104 and configured to puncture the blood vessel 200 to retrieve blood or inject medicine into the blood vessel 200. The vessel entry device 130 includes a flexible cylindrical body 132 defining a central lumen 134 or passage extending from a first end 136 to a second end 138 of the cylindrical body 132. It may be appreciated that an outer diameter of the cylindrical body 132 is substantially equal to an inner diameter of the conduit 104. Further, the vessel entry device 130 includes a needle 140 arranged coaxially with the central lumen 134 and extending outwardly from the second end 138 of the cylindrical body 132. The transition from the body of the vessel entry device to the puncture needle portion of the device consists of two 90 degree angles (or approximate thereof) to allow only the tip of the puncture needle and not the body of the entry device to enter the lumen of the blood vessel. The needle 140 is a hollow needle and includes a puncture tip 142 adapted to puncture only the adjacent blood vessel wall 200, to gain entry to the blood vessel lumen 143. The puncture tip 142 defines an orifice 144 to facilitate an insertion of a guidewire (not shown) inside the blood vessel 200 through the central lumen 134 and the hollow needle 140. In an embodiment, the needle 140 is integrally formed with the cylindrical body 132. Alternatively, the needle 140 may be removable engaged with the cylindrical body 132. To puncture the blood vessel 200, the vessel entry device 130 is inserted inside the conduit 104 through the first port 108. Additionally, to facilitate a holding of the vessel entry device 130, and enable an easy removal and insertion of the needle 140 from the conduit 104, the vessel entry device 130 includes a pair of grab arms 146, 148 arranged at the first end 136 of the cylindrical body 132. As shown, the grab arms 146, 148 extend radially outwardly of the cylindrical body 132 and are arranged diametrically opposite to each other. Additionally, the cylindrical body 132 defines a female leur lock 150 defined at the first end 136 of the cylindrical body 132 and arranged surrounding an outlet port 152 of the central lumen 134 to enable an engagement of a leur lock with the cylindrical body 132 to close the outlet port 152 when desired.

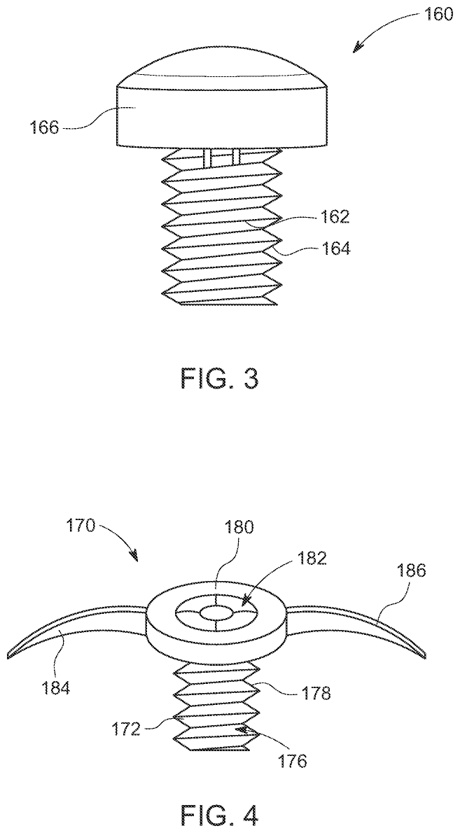

Moreover, referring to FIG. 3, the vascular access system 100 includes a cap 160 to close the first port 108 of the passage 118 (i.e., the conduit 104). As shown, the cap 160 includes a spindle portion 162 having outer threads 164 adapted to engage with the internal threads 126 of the access conduit 104, and a cover portion 166 adapted to remain outside the conduit 104, covering the first port 108. An outer diameter of the spindle portion 162 is substantially equal to the inner diameter of the access conduit 104, while an outer diameter of the cover portion 166 is greater than an outer diameter of the access conduit 104. To perform the medical procedure, for example, the dialysis, the cap 160 is removed from the conduit 104.

Additionally, referring to FIG. 4, the vascular access system 100 includes a self-sealing valve 170 adapted to be engaged with conduit 104 during the medical procedure. The valve 170 includes a hollow cylindrical portion 172 defining a passageway 176 adapted to be arranged coaxially with the passage 118 of the conduit, and external threads 178 defined at an outer surface of the cylindrical portion 172. The cylindrical portion 172 is arranged inside the conduit 104 when the valve 170 is in engagement with the conduit 104, and has an outer diameter substantially equal to the inner diameter of the conduit 104. The valve 170 is engaged with conduit 104 by engaging the external threads 178 of the cylindrical portion 172 with the inner threads 126 of the conduit 104. Moreover, the valve 170 includes a flange portion 180 extending longitudinally outwardly from the cylindrical portion 172. The flange portion 180 includes a flexible membrane 182 arranged substantially centrally to the flange portion 180 and covering the passageway 176. The flexible membrane(s) 182 is adapted to open under a force to allow an insertion or removal of medical device or equipment, while being bias to a closed position to prevent a leakage of fluid or blood from the conduit 104. Accordingly, the membrane(s) 182 is biased to the closed position to close the access of the central passage 118 of the access conduit 104 through the first port 108 and central passageway 176 of the valve 170. Moreover, the membrane(s) 182 selectively allows a passage of the vessel entry device 130, or the medical device (i.e., catheter) or the guidewire upon application of an insertion force. However, it may be appreciated that the membrane(s) 182, being biased to the closed position, closes any gap between the vessel entry device 130 or the guidewire or the catheter, or any other such device, and the flange portion 180 once the device is inserted inside the conduit 104. To facilitate the engagement and removal of the valve 170 with the conduit 104, the valve 170 includes a pair of grab arms 184, 186 extending radially outwardly from the flange portion 180 and arranged diametrically opposite to each other.

A method for performing a medical procedure, for example, the hemodialysis, using the vascular access system 100 is now described. The method includes attaching the access device 102 with the blood vessel 200. For so doing, a medical practitioner makes a small incision at a suitable position to access the blood vessel 200. The incision may extend from the skin surface 500 to the blood vessel 200 through the subcutaneous tissues 400. Thereafter, the medical practitioner extends the access device 102 partially through a smaller adjacent skin incision up to the blood vessel 200 exposed by the initial incision such that the second end 110 of the conduit 104 abuts the outer surface 202 of the blood vessel 200, and the second portion 116 of the conduit 104 extends, at least, from the outer surface 202 of the blood vessel 200 to the skin surface 500 and the first portion 114 of the conduit 104 extends outwardly of the skin surface 500. In an embodiment, the medical practitioner attaches the second end 110 of the conduit 104 to the outer surface 202 by suturing the second end 110 with the outer surface 202 of the blood vessel 200. Further, a sealing substance is optionally applied along the junction of the access conduit 104 with the outer surface 202 of the blood vessel 200 to form a cohesive joint/connection.

Thereafter, the method includes engaging the valve 170 to the first end 106 of the conduit 104. For so doing, the medical practitioner inserts the cylindrical portion 172 inside the first portion 114 through the first port 108, and engages the external threads 178 of the cylindrical portion 172 with the inner threads 126 of the access device 102. It may be appreciated that the membrane(s) 182 is arranged in the closed position during the insertion of the cylindrical portion 172 inside the conduit 104 through the first port 108. Thereafter, the method includes inserting the vessel entry device 130 inside the conduit 104 to puncture only the anterior wall of the blood vessel 200 which is directly attached the second end 110 of the conduit 104 so as to extract blood from the blood vessel 200 and/or to inject medicine inside the blood vessel 200 and to insert a leading edge of a flexible guidewire into the lumen of the blood vessel 200. For so doing, the medical practitioner holds the vessel entry device 130 by holding the grab arms 146, 148, and inserts the needle 140 and the cylindrical body 132 through the flexible membrane(s) 182, causing an opening of the membrane. 182 As the medical practitioner extends the cylindrical body 132 and the needle 140 inside the conduit 104, the tip 142 of the needle 140 contacts the outer surface 202 of the blood vessel 200 through the second port 112 of the conduit 104, and punctures and enters the blood vessel 200 with only the tip 142 of the needle 140 entering the blood vessel 202 as the vessel entry device 130 is pushed further inside the conduit 104.

Upon puncturing the blood vessel 200 via the needle 140, a guidewire (not shown) is inserted into the central lumen 134 through the outlet port 152, and is extended inside the blood vessel 200 through the central lumen 134 and the orifice 144 of the needle 140. Upon insertion of the guidewire inside the blood vessel 200, the vessel entry device 130 may be removed, while keeping the guidewire in position. The guidewire tip is then advanced to the appropriate location(s) in the vascular system of the body 300 using common imaging, catheter and other endovascular techniques known in the art. Thereafter, the medical practitioner may insert a suitable medical device, for example, a catheter, over the guidewire inside the conduit 104 through the valve 170 such that a portion (i.e., a tip) of the medical device extends inside the blood vessel 200 through the puncture formed in the blood vessel 200 and can then be advanced to a desired location for treatment, monitoring, sampling or administration of various agents, treatments or devices.

Subsequent to the removal of the blood from the blood vessel 200 or injection of the medicine into the blood vessel 200 or performing the desired medical procedure, the medical device and the guidewire are extracted and removed from the conduit 104. Upon removal of the guidewire and the medical device, the flexible membrane 182 is automatically closed, thereby the valve 170 prevents any leakage of the blood through the conduit 104. Prior to removing the guidewire and the medical device, the conduit 104 (i.e., passage 118) is flushed with suitable chemicals (e.g., antibiotics, anti-coagulants). Following the removal of the medical device and the guidewire, the first port 108 is closed by removing the valve 170 and engaging the cap 160 with the conduit 104. The blood vessel 200 puncture site is allowed to close by contraction and tissue healing, thereby separating the access device 102 from the circulation until subsequent utilization. When not in use the access device 102 is isolated from blood vessel 200 by a thin layer of vessel mural thrombus (immediately following use) and thereafter by the healed blood vessel wall. Because of the separation of the access device 103 from the blood vessel lumen, the access device can be left in place for an extended length of time, if needed, and removed as desired by a simple outpatient surgical procedure under local anesthesia.

While there have been described above the principles of the present invention in conjunction with an apparatus for harvesting of a subcutaneous blood vessel and a method therefore, it is to be understood that the foregoing description is made only by way of example and not as a limitation to the scope of the invention. Particularly, it is recognized that the teachings of the foregoing disclosure will suggest other modifications to those persons skilled in the relevant art. Such modifications may involve other features that are already known per se and which may be used instead of or in addition to features already described herein. Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The Applicant hereby reserves the right to formulate new claims to such features and/or combinations of such features during the prosecution of the present application or of any further application derived therefrom.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the present disclosure and various embodiments with various modifications as are suited to the particular use contemplated.

What is claimed is:

1. A vascular access system for accessing a blood vessel of a body, the vascular access system comprising:
an access device including
an access conduit having
a first end and a second end arranged opposite to the first end, the second end is adapted to be arranged outside the blood vessel and connected to an outer surface of the blood vessel,
a first longitudinal portion extending from the first end towards the second end and adapted to be arranged outside the body to facilitate an access of the access conduit, and
a second longitudinal portion extending from the first longitudinal portion to the second end and adapted to be arranged inside the body, and
a stent encapsulated inside the second longitudinal portion of the access conduit to prevent a collapsing of the access conduit; and
a vessel entry device adapted to extend, at least partially, inside the access conduit to puncture the blood vessel, the vessel entry device including
a cylindrical body defining a central lumen extending from a first end to a second end of the cylindrical body, and
a needle coaxially arranged relative to the cylindrical body and extending outwardly from the second end of the cylindrical body, wherein the needle includes a puncture tip to puncture the blood vessel.

2. The vascular access system of claim 1, wherein the access conduit includes a sheath wrapped around an outer surface of the access conduit and extending at least along a portion of a length of the access conduit.

3. The vascular access system of claim 1, wherein the entry device includes a pair of grab arms arranged at the first end of the cylindrical body and extending radially outwardly from the cylindrical body.

4. The vascular access system of claim 1 further including a valve adapted to be attached to the access conduit and cover a first port of the access conduit, the valve includes a flexible membrane biased in a closed position to close the first port of the access conduit.

5. The vascular access system of claim 4, wherein the vessel entry device is inserted inside the access conduit through the flexible membrane of the valve to puncture the blood vessel.

6. The vascular access system of claim 4, wherein the valve includes a cylindrical portion defining a central passageway, wherein an outer surface of the cylindrical portion defines external threads adapted to facilitate the attachment of the valve with the access conduit.

7. The vascular access system of claim 6, wherein the flexible membrane is arranged at one end of the cylindrical portion to close the central passageway.

8. The vascular access system of claim 1 further including a cap adapted to extend, at least partially, inside the access conduit to close the access conduit, wherein the cap is attached to the access conduit upon completion of the medical procedure.

9. A vascular access system for accessing a blood vessel of a body, the vascular access system comprising:
an access device including
an access conduit having
a first end and a second end adapted to be arranged outside the blood vessel and connected to an outer surface of the blood vessel,
a first longitudinal portion extending from the first end towards the second end and adapted to be arranged outside the body to facilitate an access of the access conduit, and
a second longitudinal portion extending from the first longitudinal portion to the second end and adapted to be arranged inside the body, and
a stent encapsulated inside the second longitudinal portion to prevent a collapsing of the conduit.

10. The vascular access system of claim 9 further including a vessel entry device adapted to extend, at least partially, inside the access conduit to puncture and enter the lumen of the blood vessel, the vessel entry device including
a cylindrical body defining a central lumen extending from a first end to a second end of the cylindrical body, and
a needle coaxially arranged relative to the cylindrical body and extending outwardly from the second end of the cylindrical body, wherein the needle includes a puncture tip to puncture and enter the lumen of the blood vessel.

11. The vascular access system of claim 9, wherein the access conduit includes a sheath wrapped around an outer surface of the access conduit and extending at least along a portion of a length of the access conduit.

12. The vascular access system of claim 10 further including a valve adapted to be attached to the access conduit to selectively provide an access of the access conduit, the valve includes a flexible membrane biased in a closed position to close a first port of the access conduit.

13. The vascular access system of claim 12, wherein the vessel entry device is inserted inside the access conduit through the flexible membrane of the valve to puncture the blood vessel.

14. The vascular access system of claim 12, wherein the valve includes a cylindrical portion defining a central passageway, wherein an outer surface of the cylindrical portion defines external threads adapted to facilitate the attachment of the valve with the access conduit.

15. The vascular access system of claim 14, wherein the flexible membrane is arranged at one end of the cylindrical portion to close the central passageway.

16. The vascular access system of claim 9 further including a cap adapted to extend, at least partially, inside the conduit to close the access conduit, wherein the cap is attached to the access conduit upon completion of the medical procedure.

17. A method for performing a medical procedure, comprising:
attaching an access device to an outer surface of a blood vessel, wherein the access device includes
an access conduit having a first end, a second end arranged opposite to the first end, a first longitudinal portion extending from the first end towards the second end, and a second longitudinal portion extending from the first longitudinal portion to the second end, and
a stent encapsulated inside the second longitudinal portion of the access conduit to prevent a collapsing of the conduit,
wherein the access device is attached to the blood vessel by arranging the second longitudinal portion inside the body and first longitudinal portion outside the body, wherein the second end of the access conduit is attached to the outer surface of the blood vessel;
engaging a valve at the first end of the access conduit, wherein the valve includes a flexible membrane biased to close a central passage of the access conduit and selectively provides an access of the access conduit; and
inserting a vessel entry device having a needle through the membrane and the central passage of the access conduit to puncture the blood vessel.

18. The method of claim 17 further including
extending a guidewire inside the vessel entry device to the blood vessel, wherein guidewire extends from an outside of the valve to the blood vessel through the vessel entry device,
removing the vessel entry device from the access conduit through the valve while retaining the guidewire inside the access conduit, and
inserting a medical device over the guidewire inside the access conduit and extending a portion of the medical device inside the blood vessel through the puncture formed in the blood vessel and advancing the medical device to a desired location in a vascular system of the body.

19. The method of claim 18 further including
removing the medical device and the guidewire upon completion of the medical procedure,
disengaging the valve from the access conduit, and
engaging a cap to the first end of the access conduit to plug the access conduit.

20. The method of claim 19 further including flushing the access conduit with one or more chemicals before engaging the cap to the access conduit.
